(19) United States
(12) Patent Application Publication
(10) Pub. No.: US 2023/0077192 A1
(43) Pub. Date: Mar. 9, 2023

(54) DIGITAL TWIN-BASED SIMULATION OF CARRIER PROPAGATION

(71) Applicant: INTERNATIONAL BUSINESS MACHINES CORPORATION, ARMONK, NY (US)

(72) Inventors: Saraswathi Sailaja Perumalla, Visakhapatnam (IN); Venkata Vara Prasad Karri, Visakhapatnam (IN); Sarbajit K. Rakshit, Kolkata (IN); Akash U. Dhoot, Pune (IN); Ravi Vadlamani, Hyderabad (IN)

(21) Appl. No.: 17/447,077

(22) Filed: Sep. 8, 2021

(51) Int. Cl. G16H 50/80; G06F 30/20

(52) U.S. Cl. CPC G16H 50/80; G06F 30/20

(57) ABSTRACT

According to one embodiment, a method, computer system, and computer program product for digital twin carrier simulation is provided. The embodiment may include identifying an occurrence of a contamination event. The embodiment may also include generating a digital twin of an area within a preconfigured distance of the contamination event. The embodiment may further include identifying one or more modes of spread for a contaminant released during the contamination event. The embodiment may also include performing a digital twin simulation of the area using the generated digital twin and the one or more identified modes of spread. The embodiment may further include calculating a contaminant propagation pattern based on the digital twin simulation.

### BACKGROUND

The present invention relates generally to the field of computing, and more particularly to digital twin modeling.

A digital twin relates to a virtual model designed to closely correspond with a physical object. The physical object may, typically, be outfitted with various sensors to capture data related to key functionality areas. In turn, the affixed sensors gather data regarding aspects of the physical object's performance and characteristics over time. This key information can be used to recreate the physical object in a digital form (i.e., a digital model of the physical object) that allows for exposure to and analysis of the impact on performance and possible improvements to the physical object when exposed to different simulation environments.

### SUMMARY

According to one embodiment, a method, computer system, and computer program product for digital twin carrier simulation is provided. The embodiment may include identifying an occurrence of a contamination event. The embodiment may also include generating a digital twin of an area within a preconfigured distance of the contamination event. The embodiment may further include identifying one or more modes of spread for a contaminant released during the contamination event. The embodiment may also include performing a digital twin simulation of the area using the generated digital twin and the one or more identified modes of spread. The embodiment may further include calculating a contaminant propagation pattern based on the digital twin simulation.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment.

FIG. 2 illustrates an operational flowchart for a digital twin carrier simulation process according to at least one embodiment.

FIG. 3 is a block diagram of internal and external components of computers and servers depicted in FIG. 1 according to at least one embodiment.

FIG. 4 depicts a cloud computing environment according to an embodiment of the present invention.

FIG. 5 depicts abstraction model layers according to an embodiment of the present invention.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

It is to be understood that the singular forms “a,” “an,” and “the” include plural referents unless the context clearly dictates otherwise. Thus, for example, reference to “a component surface” includes reference to one or more of such surfaces unless the context clearly dictates otherwise.

Embodiments of the present invention relate to the field of computing, and more particularly to digital twin modeling. The following described exemplary embodiments provide a system, method, and program product to, among other things, utilize digital twin modelling to simulate the transmission of pathogens throughout a preconfigured area. Therefore, the present embodiment has the capacity to improve the technical field of digital twin modelling by expanding the typical application of digital twin modelling from recreation of a specific object and performance and characteristics of the object in a controlled setting to being a preconfigured area and the transmission of pathogenic carriers throughout the area.

As previously described, a digital twin relates to a virtual model designed to closely correspond with a physical object. The physical object may, typically, be outfitted with various sensors to capture data related to key functionality areas. In turn, the affixed sensors gather data regarding aspects of the physical object's performance and characteristics over time. This key information can be used to recreate the physical object in a digital form (i.e., a digital model of the physical object) that allows for exposure to and analysis of the impact on performance and possible improvements to the physical object when exposed to different simulation environments.

In the field of disease transmission, understanding propagation patterns of airborne pathogens is vitally important to identifying possible contamination. For example, droplets propelled through the air that contain a virus can spread quickly throughout an area if the airborne propagation patterns are not fully understood. Determining the propagation patterns may be difficult under current means may be difficult since few practical method exist to simulate actual patterns of propagation for airborne droplets, or other contaminants. As such, it may be advantageous to, among other things, utilize digital twin simulations to identify how various contaminant types propagate in specific areas based on conditions and characteristics.

According to at least one embodiment, similar to a typical digital twin model, various sensors may be utilized to gather data regarding a subject. However, unlike a typical digital twin model, the object of interest may be a preconfigured area, such as a room, a building interior, or a city. In order to accurately capture relevant information to propagation patterns of a contaminant, such as a virus, bacteria, or other pathogen, various sensors may be utilized to capture data relating to vehicle traffic, air patterns, water patterns, and human movement. Using this data, a digital twin engine may perform data analysis to determine contaminant spread within the preconfigured area to allow for a proper mitigation and severity prediction of affected individuals.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method, and program product to identify contaminant spread in a preconfigured area or space using digital twin-based techniques.

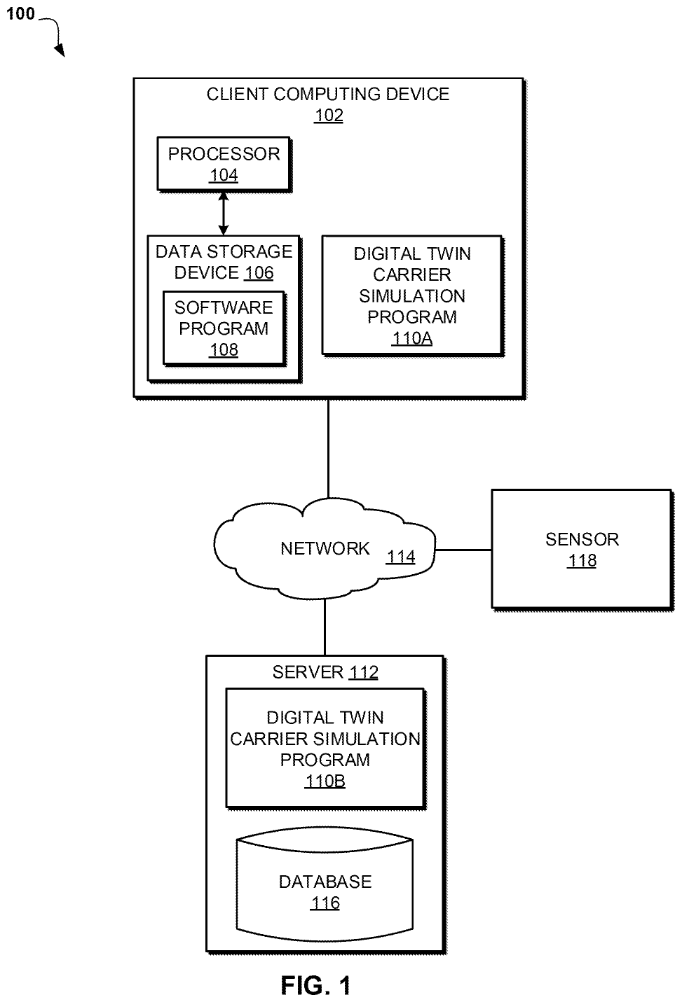

Referring to FIG. 1, an exemplary networked computer environment 100 is depicted, according to at least one embodiment. The networked computer environment 100 may include client computing device 102, a server 112, and one or more sensors 118 interconnected via a communication network 114. According to at least one implementation, the networked computer environment 100 may include a plurality of client computing devices 102, servers 112 and sensors 118, of which only one of each is shown for illustrative brevity. Additionally, in one or more embodiments, the client computing device 102 and server 112 may each individually host a digital twin carrier simulation program 110A, 110B. In one or more other embodiments, the digital twin carrier simulation program 110A, 110B may be partially hosted on both client computing device 102 and server 112 so that functionality may be separated between the devices.

The communication network 114 may include various types of communication networks, such as a wide area network (WAN), local area network (LAN), a telecommunication network, a wireless network, a public switched network and/or a satellite network. The communication network 114 may include connections, such as wire, wireless communication links, or fiber optic cables. It may be appreciated that FIG. 1 provides only an illustration of one implementation and does not imply any limitations with regard to the environments in which different embodiments may be implemented. Many modifications to the depicted environments may be made based on design and implementation requirements.

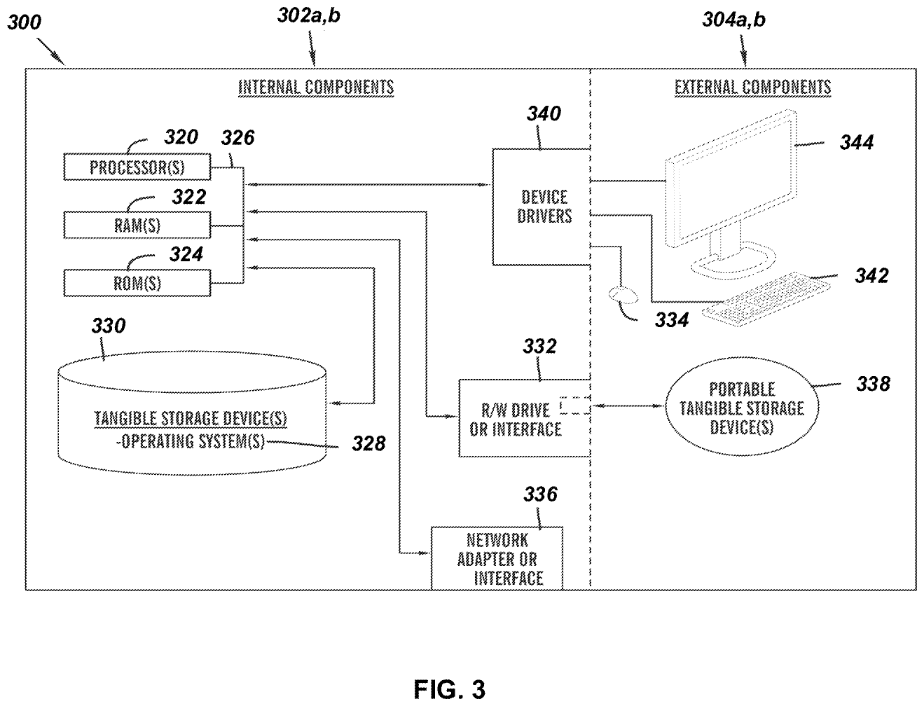

Client computing device 102 may include a processor 104 and a data storage device 106 that is enabled to host and run a software program 108 and a digital twin carrier simulation program 110A, receive data from one or more sensors, such as sensor 118, and communicate with the server 112 via the communication network 114, in accordance with one embodiment of the invention. In one or more other embodiments, client computing device 102 may be, for example, a mobile device, a telephone, a personal digital assistant, a netbook, a laptop computer, a tablet computer, a desktop computer, or any type of computing device capable of running a program and accessing a network. As previously described, one client computing device 102 is depicted in FIG. 1 for illustrative purposes, however, any number of client computing devices 102 may be utilized. As will be discussed with reference to FIG. 3, the client computing device 102 may include internal components 302a and external components 304a, respectively.

The server computer 112 may be a laptop computer, netbook computer, personal computer (PC), a desktop computer, or any programmable electronic device or any network of programmable electronic devices capable of hosting and running a digital twin carrier simulation program 110B and a database 116 and communicating with the client computing device 102 via the communication network 114, in accordance with embodiments of the invention. As will be discussed with reference to FIG. 3, the server computer 112 may include internal components 302b and external components 304b, respectively. The server 112 may also operate in a cloud computing service model, such as Software as a Service (SaaS), Platform as a Service (PaaS), or Infrastructure as a Service (IaaS). The server 112 may also be located in a cloud computing deployment model, such as a private cloud, community cloud, public cloud, or hybrid cloud.

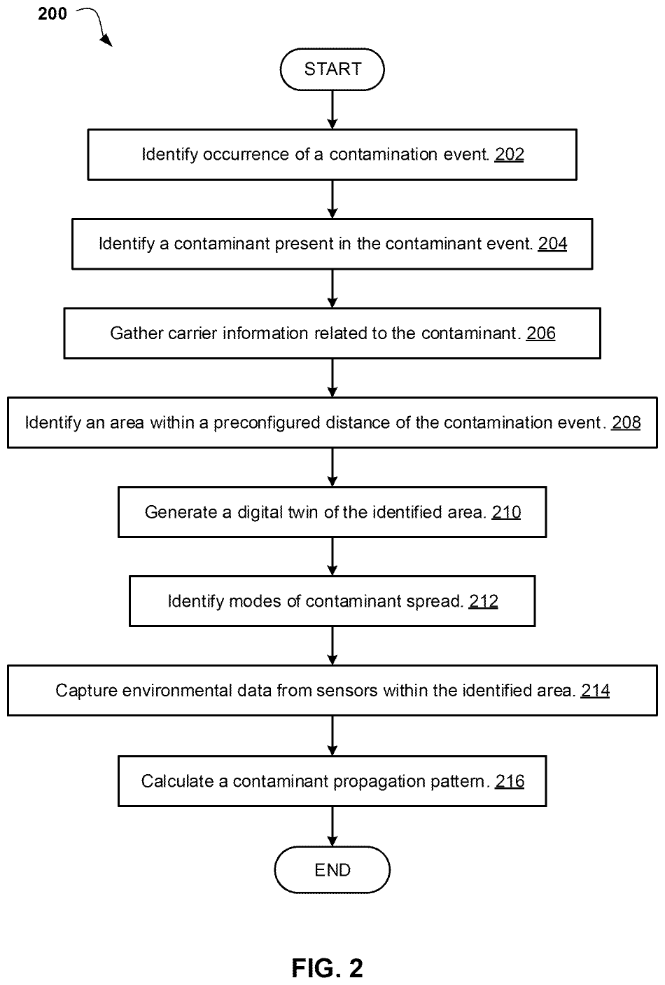

According to the present embodiment, the digital twin carrier simulation program 110A, 110B may be capable of capture various characteristics relating to the contaminant, such as transmissibility, impact to human health, lifespan, and morbidity, and utilizing digital twin simulation techniques to identify how different types of contaminations, such as pathogens or other biohazardous materials, can propagate in an preconfigured area, such as a room, building, city block, or neighborhood, considering the mobility pattern of various entities, such as people, vehicles, water, and air. Accordingly, the digital twin carrier simulation program 110A, 110B may proactively be identifying a propagation pattern of the contaminants so that, based on identified behavior of the contaminant, the digital twin carrier simulation program 110A, 110B can determine how the contaminant will likely spread throughout the preconfigured area. In at least one embodiment, digital twin carrier simulation program 110A, 110B may calculate an impact of a contaminant on the preconfigured area in terms of resources needed to remove the contaminant and medically treat any living organisms impacted by the contaminants release. The digital twin carrier simulation method is explained in further detail below with respect to FIG. 2.

Referring now to FIG. 2, an operational flowchart illustrating a digital twin carrier simulation process 200 is depicted according to at least one embodiment. At 202, the digital twin carrier simulation program 110A, 110B identifies the occurrence of a contamination event. A contamination event may relate to any event that releases or identifies a contaminant within a surrounding environment, such as identifying an individual becoming infected with a pathogen, or another transmissible agent, or a hazardous materials spill capable of continued spread. A pathogen may include algae, bacteria, fungi, prions, viroids, viruses, and parasites. The digital twin carrier simulation program 110A, 110B may identify the occurrence of the contamination event through various input methods, such as natural language processing, image processing, and speech-to-text. For example, the digital twin carrier simulation program 110A, 110B may identify a vehicle accident that releases biohazardous materials as a contamination event when the accident is observed via live camera feeds throughout a smart city. In a similar example, the digital twin carrier simulation program 110A, 110B may identify an individual describing symptoms of a transmissible airborne disease to a call-in emergency hotline as a contamination event. The digital twin carrier simulation program 110A, 110B may also identify a contamination event by manual user input through a graphical user interface or a peripheral device connected to or associated with a user device, such as client computing device 102. In yet another embodiment, the digital twin carrier simulation program 110A, 110B may identify the occurrence of the contaminant event through distributed sensors capable of detecting the presence of a contaminant. For example, sensors capable of detecting the presence of radioactive material may be distributed through a city and, when an event occurs that releases radioactive material, the sensors may transmit a notification to the digital twin carrier simulation program 110A, 110B indicating the presence of a contamination event.

Then, at 204, the digital twin carrier simulation program 110A, 110B identifies the contaminant. Based on the information gathered when identifying the occurrence of the contamination event, the contaminant may be known to the digital twin carrier simulation program 110A, 110B or the digital twin carrier simulation program 110A, 110B may need to make a prediction as to the identity of the contaminant involved in the contamination event. For example, in the previous example of a vehicle accident, the biohazardous material may be identified as gasoline, oil, or another petroleum-based product using known image recognition techniques. In another embodiment, the digital twin carrier simulation program 110A, 110B may attempt to predict the contaminant from information provided, such as a caller to an emergency hotline describing specific illness symptoms. In at least one embodiment, when the digital twin carrier simulation program 110A, 110B makes a prediction as to the identity of the contaminant, the digital twin carrier simulation program 110A, 110B may rank likely contaminant identities based on probability or may proceed with producing a digital twin simulation for each possible identity. In at least one other embodiment, the digital twin carrier simulation program 110A, 110B may also identify a contaminant by manual user input through a graphical user interface or a peripheral device connected to or associated with a user device, such as client computing device 102.

Then, at 206, the digital twin carrier simulation program 110A, 110B gathers carrier information related to the contaminant. Using various sources, the digital twin carrier simulation program 110A, 110B may gather information related to characteristics of the contaminant so as to identify one or more carriers of the contaminant. A carrier may be identified as any entity or substance that is capable of hosting, transmitting, or otherwise moving a contaminant from one location or entity to another location or entity. For infectious disease-related or pathogenic contaminants, the digital twin carrier simulation program 110A, 110B may identify carrier characteristics as reservoirs of the pathogen (e.g., animal populations, soil, water, and inanimate object or materials), pathogen lifespan, contact transmissibility, vector transmissibility, vehicle transmissibility, and healthcare-associated infections. Contact transmission may relate to whether a contaminant can be directly or indirectly transmitted through physical contact with either an infected host (i.e., direct transmission) or through contact with a fomite with which an infected host has previously made contact (i.e., indirect transmission). Vector transmission may occur when a living organism carries an infectious agent on its body (i.e., mechanical transmission) or as an infection host itself (i.e., biological transmission) either of which may cause an infection to another organism. Vehicle transmission may occur when a substance, such as soil, water, or air, carries an infectious agent to a new host. Healthcare-associated infections (HAIs), or nosocomial infections, may be infections acquired in a clinical setting. Transmission of HAIs may be facilitated by medical intervention and may impact high concentrations of susceptible, immunocompromised individuals in clinical settings. In at least one embodiment, the digital twin carrier simulation program 110A, 110B may receive updated information of various pathogens to improve identification of potential carriers. For example, when a new infectious disease is identified, very little information is initially known about the disease. However, as more information is discovered and becomes available, the digital twin carrier simulation program 110A, 110B may update the carrier information accordingly.

In at least one embodiment, the digital twin carrier simulation program 110A, 110B may identify carrier information of any non-infectious disease contaminant that is hazardous to living organisms or whose presence and spread is otherwise detrimental or unwanted. For example, biohazardous materials may be hazardous to living organisms and unrestrained spread of such materials, such as during an accidental spill, may be detrimental to living organisms. Similarly, the spread of an otherwise non-hazardous substance may not be dangerous for the health of living organisms but a digital twin simulation of the potential spread of the substance may be financially beneficial for cleanup efforts.

Next, at 208, the digital twin carrier simulation program 110A, 110B identifies an area within a preconfigured distance of the contamination event. Upon the occurrence of the contamination event and identification of potential carriers of the contaminant, the digital twin carrier simulation program 110A, 110B may generate an area within a preconfigured distance of the contamination event that is most likely to be impacted by after affects of the contamination event. For example, if the contamination event is the spillage of radioactive materials, the digital twin carrier simulation program 110A, 110B may define the preconfigured area as the area the most likely to be impacted by the effects of the spill. The digital twin carrier simulation program 110A, 110B may also define the area based on how the type of contaminant is likely to spread. For example, if the contaminant is an airborne disease, the digital twin carrier simulation program 110A, 110B may define the area as an airspace around the location of the contaminant event. However, if the contaminant is an oil spill, the digital twin carrier simulation program 110A, 110B may define the area as the ground surface, surface water bodies, waterways, and ground water within a preconfigured distance of the contamination event.

Then, at 210, the digital twin carrier simulation program 110A, 110B generates a digital twin of the identified area. As previously described, a digital twin is a virtual model designed to closely correspond with a physical object. Typically, the physical object is outfitted with various sensors to capture data related to key functionality areas. In at least one embodiment, the digital twin carrier simulation program 110A, 110B may use various data sources to generate a virtual model of the identified area. The various sources may comprise maps, such maps relating to geographic, topology, geology, roadways, public transportation, and water ways; structural layouts, airflow patterns, and weather forecasts.

Next, at 212, the digital twin carrier simulation program 110A, 110B identifies one or more modes of contaminant spread within the identified area. The digital twin carrier simulation program 110A, 110B may make this determination based on historical data of known modes of contaminant spread, or carriers, from various sources of information, such as journal articles or other informational databases, such as database 116. While creating and performing the digital twin simulation, the digital twin carrier simulation program 110A, 110B may determine how different carriers are individually traversing throughout the identified area which may lead to propagation of the contaminant. In terms of the propagation of carriers of a contaminated disease, this determination may include identifying the presence of a disease and the presence of a carrier capable of transmitting the disease, such as aerosols carrying an airborne disease through an enclosed office space. Additionally, when two or more carriers are present, the digital twin carrier simulation program 110A, 110B may make a correlation between the two carriers and how the presence of each may impact the propagation through an area.

Then, at 214, the digital twin carrier simulation program 110A, 110B captures environmental data from sensors within the identified area. The digital twin carrier simulation program 110A, 110B may be capable of determining the speed and direction of propagation of various types of carriers in the area. For example, in the event the contaminant is an airborne disease, the digital twin carrier simulation program 110A, 110B may identify a carrier as the airflow in a building and the speed and direction of propagation may be the speed and direction of the air flow. Based on the types of carriers and the identified area, the digital twin carrier simulation program 110A, 110B may utilize various data sources to simulate the transmission of the contaminant throughout the area. The various data sources may also include an Internet of Things (IoT) feed from sensors at various locations around the identified area that may aid in the measurement of crowd movements, public transportation system movements, private passenger vehicle movements, drainage system flowrates and directions, geographic landscape information (e.g., river flow volume, rate, and direction), air flow-related information (e.g., air flow rate, volume, and direction), and current and/or future weather conditions. Furthermore, the digital twin carrier simulation program 110A, 110B may identify restrictions to the contaminant spread within the identified area, such as natural barriers or the presence of anaphylactic agents preventing further contaminant spread. Additionally, the digital twin carrier simulation program 110A, 110B may identify activities occurring in the identified area that may impact, either positively or negatively, the contaminant propagation. For example, the presence of venues historically linked with large gatherings may result in an increased propagation of a transmissible disease. Furthermore, the digital twin carrier simulation program 110A, 110B may consider environmental conditions that can also affect contaminant propagation, such as the impact of high humidity on infectious disease-carrying aerosols.

Next, at 216, the digital twin carrier simulation program 110A, 110B calculates a contaminant propagation pattern using the generated digital twin based on the modes of contaminant spread and the captured sensor data. Based on the digital twin simulation results, the digital twin carrier simulation program 110A, 110B may identify how individual modes of contaminant spread propagates in the identified area (e.g., speed and direction of propagation) and an effect that the presence of multiple modes of contaminant spread may have on the overall contaminant propagation throughout the identified area. Using this information, the digital twin carrier simulation program 110A, 110B may identify sections of the identified area most likely to be affected by the propagation of carriers throughout the identified area. For example, the digital twin carrier simulation program 110A, 110B may identify certain areas of a smart-enabled city that are most likely to be affected in the event an infectious disease is released at a specific location. In at least one embodiment, the digital twin carrier simulation program 110A, 110B may identify entities that can be impacted through the propagation of the contaminant. For example, in the event of an infectious disease, the digital twin carrier simulation program 110A, 110B may identify businesses likely to be impacted through the digital twin-simulated propagation. Additionally, the digital twin carrier simulation program 110A, 110B may calculate a financial impact to the identified area due to the presence of the contaminant by estimating an amount of remediation necessary to remove the contaminant, medical costs associated with injuries to living organisms as a result of the contaminant's presence, and closures required to prevent further propagation of the contaminant in, and potentially out of, the identified area. The estimated financial impact may be calculated using current rates for applicable services as indicated on available repositories, such as database 116.

In at least one other embodiment, the digital twin carrier simulation program 110A, 110B may utilize anticipated weather conditions to predict the contamination propagation pattern and proactively take action so that propagation intensity of the contamination can be minimized. For example, if heavy rains are predicted by a weather forecasting service, the digital twin carrier simulation program 110A, 110B may determine flooding conditions will spread the contamination and indicate that proactive action be taken to clean and remove any contaminated objects before the forecasted weather exacerbates the contaminant spread.

It may be appreciated that FIG. 2 provides only an illustration of one implementation and does not imply any limitations with regard to how different embodiments may be implemented. Many modifications to the depicted environments may be made based on design and implementation requirements.

FIG. 3 is a block diagram 300 of internal and external components of the client computing device 102 and the server 112 depicted in FIG. 1 in accordance with an embodiment of the present invention. It should be appreciated that FIG. 3 provides only an illustration of one implementation and does not imply any limitations with regard to the environments in which different embodiments may be implemented. Many modifications to the depicted environments may be made based on design and implementation requirements.

The data processing system 302, 304 is representative of any electronic device capable of executing machine-readable program instructions. The data processing system 302, 304 may be representative of a smart phone, a computer system, PDA, or other electronic devices. Examples of computing systems, environments, and/or configurations that may represented by the data processing system 302, 304 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The client computing device 102 and the server 112 may include respective sets of internal components 302 a,b and external components 304 a,b illustrated in FIG. 3. Each of the sets of internal components 302 include one or more processors 320, one or more computer-readable RAMs 322, and one or more computer-readable ROMs 324 on one or more buses 326, and one or more operating systems 328 and one or more computer-readable tangible storage devices 330. The one or more operating systems 328, the software program 108 and the digital twin carrier simulation program 110A in the client computing device 102 and the digital twin carrier simulation program 110B in the server 112 are stored on one or more of the respective computer-readable tangible storage devices 330 for execution by one or more of the respective processors 320 via one or more of the respective RAMs 322 (which typically include cache memory). In the embodiment illustrated in FIG. 3, each of the computer-readable tangible storage devices 330 is a magnetic disk storage device of an internal hard drive. Alternatively, each of the computer-readable tangible storage devices 330 is a semiconductor storage device such as ROM 324, EPROM, flash memory or any other computer-readable tangible storage device that can store a computer program and digital information.

Each set of internal components 302 a,b also includes a R/W drive or interface 332 to read from and write to one or more portable computer-readable tangible storage devices 338 such as a CD-ROM, DVD, memory stick, magnetic tape, magnetic disk, optical disk or semiconductor storage device. A software program, such as the digital twin carrier simulation program 110A, 110B, can be stored on one or more of the respective portable computer-readable tangible storage devices 338, read via the respective R/W drive or interface 332, and loaded into the respective hard drive 330.

Each set of internal components 302 a,b also includes network adapters or interfaces 336 such as a TCP/IP adapter cards, wireless Wi-Fi interface cards, or 3G or 4G wireless interface cards or other wired or wireless communication links. The software program 108 and the digital twin carrier simulation program 110A in the client computing device 102 and the digital twin carrier simulation program 110B in the server 112 can be downloaded to the client computing device 102 and the server 112 from an external computer via a network (for example, the Internet, a local area network or other, wide area network) and respective network adapters or interfaces 336. From the network adapters or interfaces 336, the software program 108 and the digital twin carrier simulation program 110A in the client computing device 102 and the digital twin carrier simulation program 110B in the server 112 are loaded into the respective hard drive 330. The network may comprise copper wires, optical fibers, wireless transmission, routers, firewalls, switches, gateway computers and/or edge servers.

Each of the sets of external components 304 a,b can include a computer display monitor 344, a keyboard 342, and a computer mouse 334. External components 304 a,b can also include touch screens, virtual keyboards, touch pads, pointing devices, and other human interface devices. Each of the sets of internal components 302 a,b also includes device drivers 340 to interface to computer display monitor 344, keyboard 342, and computer mouse 334. The device drivers 340, R/W drive or interface 332, and network adapter or interface 336 comprise hardware and software (stored in storage device 330 and/or ROM 324).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

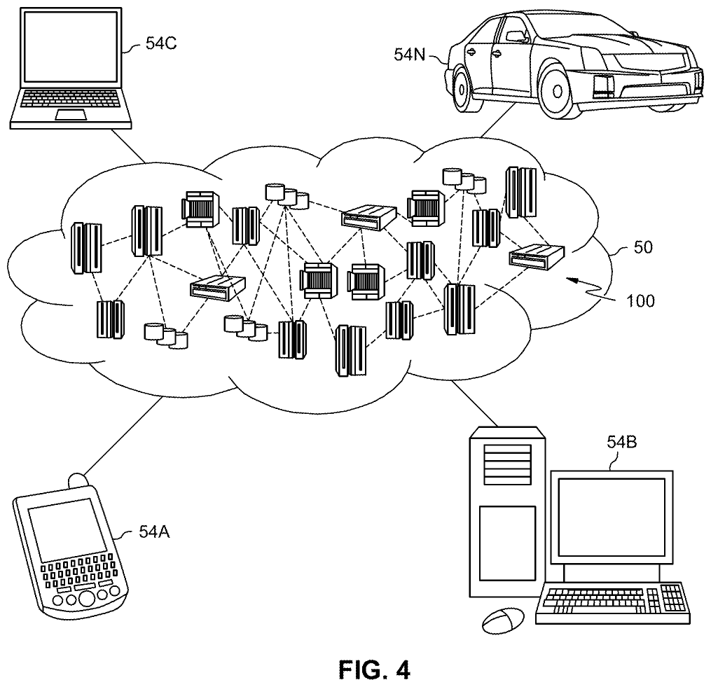

Referring now to FIG. 4, illustrative cloud computing environment 50 is depicted. As shown, cloud computing environment 50 comprises one or more cloud computing nodes 100 with which local computing devices used by cloud consumers, such as, for example, personal digital assistant (PDA) or cellular telephone 54A, desktop computer 54B, laptop computer 54C, and/or automobile computer system 54N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 100 and cloud computing environment 50 can communicate with any type of computerized device over any type of network and/or network addressable connection (e.g., using a web browser).

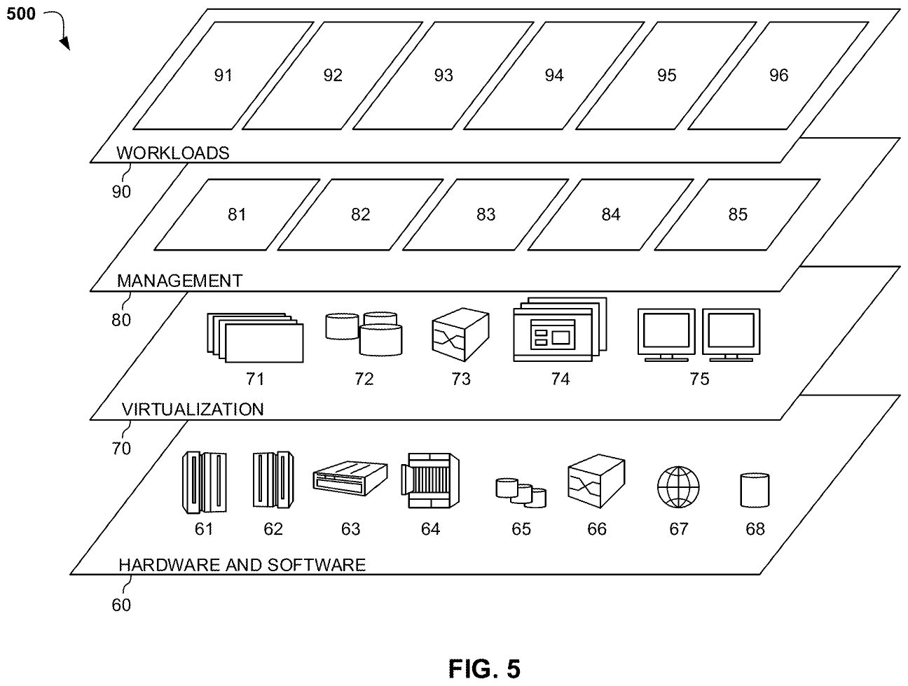

Referring now to FIG. 5, a set of functional abstraction layers 500 provided by cloud computing environment 50 is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and digital twin carrier simulation 96. Digital twin carrier simulation 96 may relate capturing data from various sensors within a preconfigured area to perform a digital twin simulation of the preconfigured area, which may identify the propagation pattern of a contaminant within the preconfigured area.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method, the method comprising:
identifying an occurrence of a contamination event;
generating a digital twin of an area within a preconfigured distance of the contamination event;
identifying one or more modes of spread for a contaminant released during the contamination event;
performing a digital twin simulation of the area using the generated digital twin and the one or more identified modes of spread; and
calculating a contaminant propagation pattern based on the digital twin simulation.

2. The method of claim 1, further comprising:
calculating a financial impact of the calculated contaminant propagation pattern.

3. The method of claim 1, further comprising:
identifying the contaminant; and
gathering a plurality of characteristic information relating to the identified carrier.

4. The method of claim 1, further comprising;
capturing environmental data from a plurality of sensors within the area.

5. The method of claim 2, wherein the financial impact is a cost associated with a presence of the contaminant as estimated by an amount of remediation necessary to remove the contaminant, medical costs associated with injuries to living organisms as a result of a presence of the contaminant, and closures required to prevent further propagation of the contaminant in the identified area.

6. The method of claim 1, wherein the contamination event is an event that releases or identifies a contaminant within a surrounding environment.

7. The method of claim 1, wherein the one or more modes of spread are entities or substances capable of hosting or transmitting the contaminant from one location or entity to another location or entity.

8. A computer system, the computer system comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
identifying an occurrence of a contamination event;
generating a digital twin of an area within a preconfigured distance of the contamination event;
identifying one or more modes of spread for a contaminant released during the contamination event;
performing a digital twin simulation of the area using the generated digital twin and the one or more identified modes of spread; and
calculating a contaminant propagation pattern based on the digital twin simulation.

9. The computer system of claim 8, further comprising:
calculating a financial impact of the calculated contaminant propagation pattern.

10. The computer system of claim 8, further comprising:
identifying the contaminant; and
gathering a plurality of characteristic information relating to the identified carrier.

11. The computer system of claim 8, further comprising;
capturing environmental data from a plurality of sensors within the area.

12. The computer system of claim 9, wherein the financial impact is a cost associated with a presence of the contaminant as estimated by an amount of remediation necessary to remove the contaminant, medical costs associated with injuries to living organisms as a result of a presence of the contaminant, and closures required to prevent further propagation of the contaminant in the identified area.

13. The computer system of claim 8, wherein the contamination event is an event that releases or identifies a contaminant within a surrounding environment.

14. The computer system of claim 8, wherein the one or more modes of spread are entities or substances capable of hosting or transmitting the contaminant from one location or entity to another location or entity.

15. A computer program product, the computer program product comprising:
one or more computer-readable tangible storage medium and program instructions stored on at least one of the one or more tangible storage medium, the program instructions executable by a processor capable of performing a method, the method comprising:
identifying an occurrence of a contamination event;
generating a digital twin of an area within a preconfigured distance of the contamination event;
identifying one or more modes of spread for a contaminant released during the contamination event;
performing a digital twin simulation of the area using the generated digital twin and the one or more identified modes of spread; and
calculating a contaminant propagation pattern based on the digital twin simulation.

16. The computer program product of claim 15, further comprising:
calculating a financial impact of the calculated contaminant propagation pattern.

17. The computer program product of claim 15, further comprising:
identifying the contaminant; and
gathering a plurality of characteristic information relating to the identified carrier.

18. The computer program product of claim 15, further comprising;
capturing environmental data from a plurality of sensors within the area.

19. The computer program product of claim 16, wherein the financial impact is a cost associated with a presence of the contaminant as estimated by an amount of remediation necessary to remove the contaminant, medical costs associated with injuries to living organisms as a result of a presence of the contaminant, and closures required to prevent further propagation of the contaminant in the identified area.

20. The computer program product of claim 15, wherein the contamination event is an event that releases or identifies a contaminant within a surrounding environment.
